(19) United States
(12) Patent Application Publication
(10) Pub. No.: US 2011/0295292 A1
(43) Pub. Date: Dec. 1, 2011

(54) VIBRATION DAMPENING OPHTHALMIC PNEUMATIC SURGICAL INSTRUMENT

(76) Inventor: Ta-Chung Hsia, Chesterfield, MO (US)

(21) Appl. No.: 13/106,016

(22) Filed: May 12, 2011

Related U.S. Application Data
(60) Provisional application No. 61/348,442, filed on May 26, 2010.

(51) Int. Cl. A61F 9/00

(52) U.S. Cl. 606/166

(57) ABSTRACT

An ophthalmic pneumatic surgical instrument 10 includes a housing 12, a tissue manipulating structure 14, a pneumatic driver 34, and a pneumatic power port 20. The pneumatic driver 34 is contained within the housing 12 for driving the tissue manipulating structure 14. The pneumatic power port 20 is formed in the housing 12 and is for attachment to tubing that will supply pneumatic power pulses to the pneumatic driver 34. The pneumatic power port includes a tubing connector 22 with a lumen 24 spanning the length of the tubing connector 22 and a length 26 of vibration dampening material surrounding as least a portion of the tubing connector 22 for reducing vibrations created by the pneumatic power pulses transmitted through the pneumatic power port.

### CROSS-REFERENCE TO RELATED APPLICATIONS

This application claims the benefit of U.S. Provisional Application No. 61,348,442, filed May 26, 2010, the entire disclosure of which is incorporated herein by reference.

### BACKGROUND

1. Field of the Invention

The present invention relates to pneumatically powered ophthalmic surgical instruments, especially vitreous cutters, scissors, and the like.

2. Description of the Prior Art

Pneumatic instruments are well known for use in ophthalmic surgery. These instruments are a common and effective means of manipulating, dissecting, and removing tissue. Typically, a pneumatic instrument has some type of driver within a housing, so that two parts of the surgical instrument can be moved relative to each other or a single part can be moved relative to a stationary part.

One well known pneumatic instrument, commonly referred to as a vitreous (vit) cutter, typically has an inner tubular cutter that is attached to and reciprocated by the driver. As the inner cutter reciprocates within a fixed outer cutter, tissue is aspirated through a port in the outer cutter and, via scissor contact with the outer cutter, the inner cutter severs the tissue that is within the port, as the inner cutter passes the port. The severed tissue is then typically aspirated to a container. Other pneumatic instrument configurations are also known. For example, the outer cutter may also move, the movement may be rotary or oscillating instead of reciprocating, or the instrument may be a scissors or a morcellator instead of a tubular cutter.

Vit cutter technology is progressively developing ever faster cutting speeds. Just a few years ago 1,500-2,000 cuts per minute was considered fast. Now, cutters of 3,000, 5,000, and 10,000 cuts per minute and possibly higher, are known. One potential drawback to such high-cut speeds are vibrations transmitted to a surgeon's finger tips. As the cut speeds increase, the pneumatic power pulses transmitted to the vit cutter driver also increase, and the force or pressure of these pulses is also greater than previously used. The increased vibrations generated by the high-speed vit cutters is an irritant to the surgeon and, during delicate surgery in the eye, especially close to the retina, potentially effects the ability of the surgeon to maintain a steady hand. Therefore, there is a need to provide a high speed ophthalmic pneumatic surgical instrument that dampens vibrations created by the high speed pneumatic power pulses.

### BRIEF DESCRIPTION OF THE DRAWINGS

FIG. 1 is a perspective view of an exemplary ophthalmic surgical instrument, in accordance with the present invention;

FIG. 2 is a perspective view of a portion of the instrument of FIG. 1;

FIG. 3 is a perspective view of a part of the instrument shown in FIG. 2;

FIG. 4 is a perspective view of a part of FIG. 3;

FIG. 5 is a perspective view of another part of FIG. 3;

FIG. 6 is a cut-away view of FIG. 3, along line 6-6;

FIG. 7 is a partial cut-away view of FIG. 2, along line 7-7; and

FIG. 8 is a perspective view of an alternate embodiment, in accordance with the present invention.

### DETAILED DESCRIPTION OF THE PREFERRED EMBODIMENT

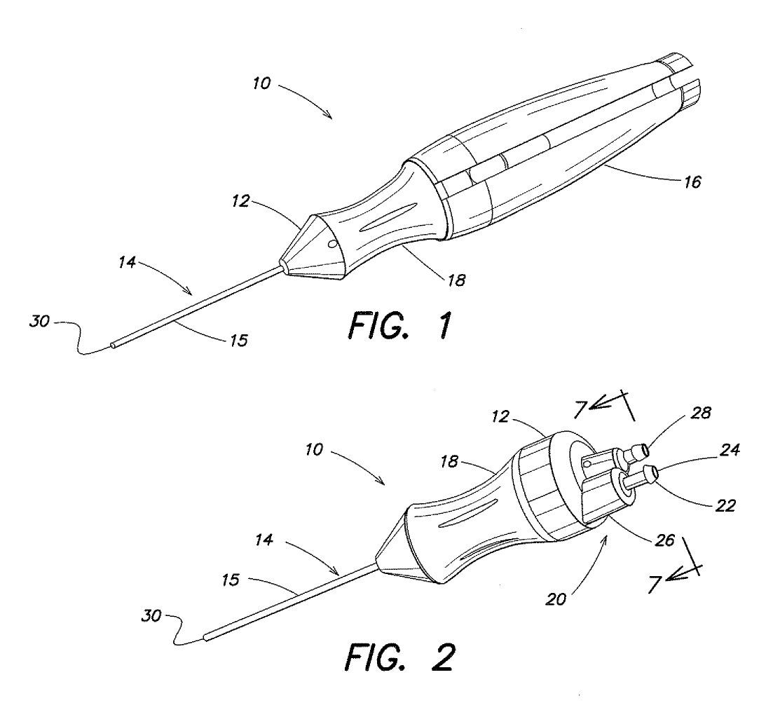

FIG. 1 shows an ophthalmic pneumatic surgical instrument 10, in this example a vit cutter, though those skilled in the art will appreciate that instrument 10 could be other known pneumatic surgical instruments used in ophthalmic surgery. Vit cutter 10 includes a housing 12 and tissue manipulating structure 14 extending from the housing 12. In this example, tissue manipulating structure 14 is a tube set including an exterior cutter 15, which is described further below in relation to FIG. 7. FIG. 1 also shows vit cutter 10, further including an extension handle 16 connected to the housing 12 for increasing the mass of the instrument 10 to further dampen vibration from pneumatic power pulses. Instrument 10 also may include a layer 18 of vibration dampening material surrounding a portion of the housing 12 for reducing vibration energy transmitted to a surgeon's fingers.

A pneumatic driver (shown and described below in relation to FIG. 7) is contained within the housing 12 for driving the tissue manipulating structure 14. A pneumatic power port 20, shown best in FIG. 2, is formed in the housing 12 and is for attachment to tubing (not shown) that will supply pneumatic power pulses to the pneumatic driver. Pneumatic power port 20 includes a tubing connector 22 with a lumen 24 spanning a length of the tubing connector 22. Pneumatic power port 20 also includes a length 26 of vibration dampening material surrounding at least a portion of the tubing connector 22 for reducing vibrations created by the pneumatic power pulses transmitted through the pneumatic power port 20. Power port 20 also includes an aspiration tubing connector 28 for allowing tissue and fluid to be aspirated through outer cutter opening 30 to be transported to a collection container (not shown), as is well known.

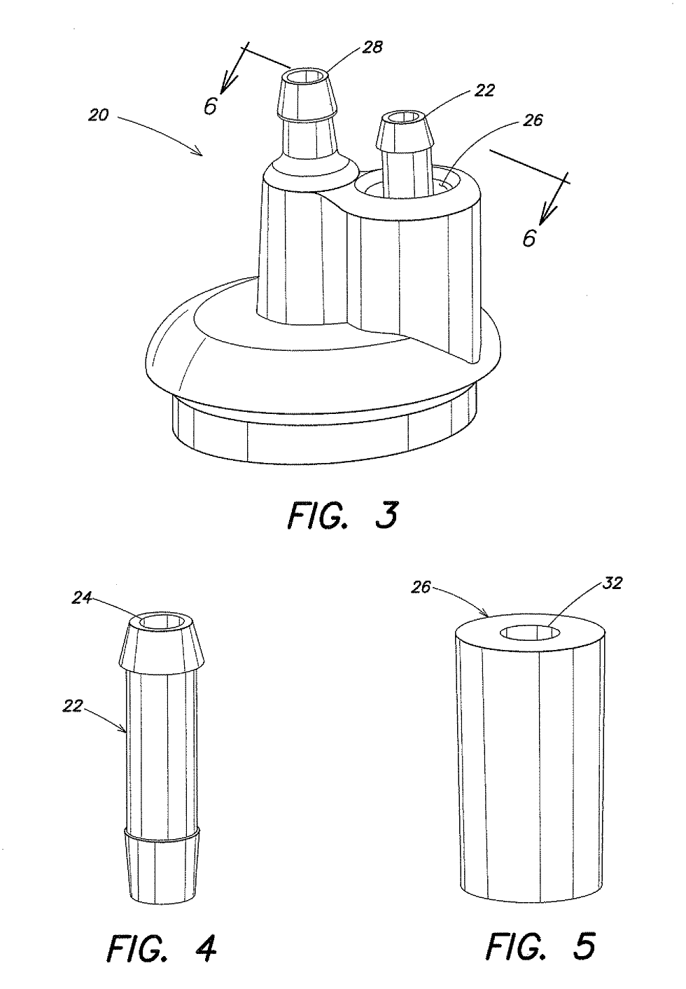

FIG. 3 shows only pneumatic power port 20. Tubing connector 22, surrounded by length 26 of vibration dampening material is more clearly seen than in FIG. 2. Tubing connector 22, as is shown in perspective in FIG. 4, is preferably formed of a material more rigid (stiff) than a material forming the housing 12 and the outer portion of pneumatic power port 20 for reducing the amplitude of vibration caused by the pneumatic power pulses. Tubing connector 22 may be a metal, such as stainless steel, brass, or other surgically acceptable metals. Typically housing 12 and power port 20 may be molded of plastic. Length 26 vibration dampening material is preferably formed of rubber or synthetic rubber, such as neoprene, ISODAMP® C-1002, or the like. Layer 18 may also be formed of similar material as length 26. Length 26 has a lumen 32 within which tubing connector 22 is placed.

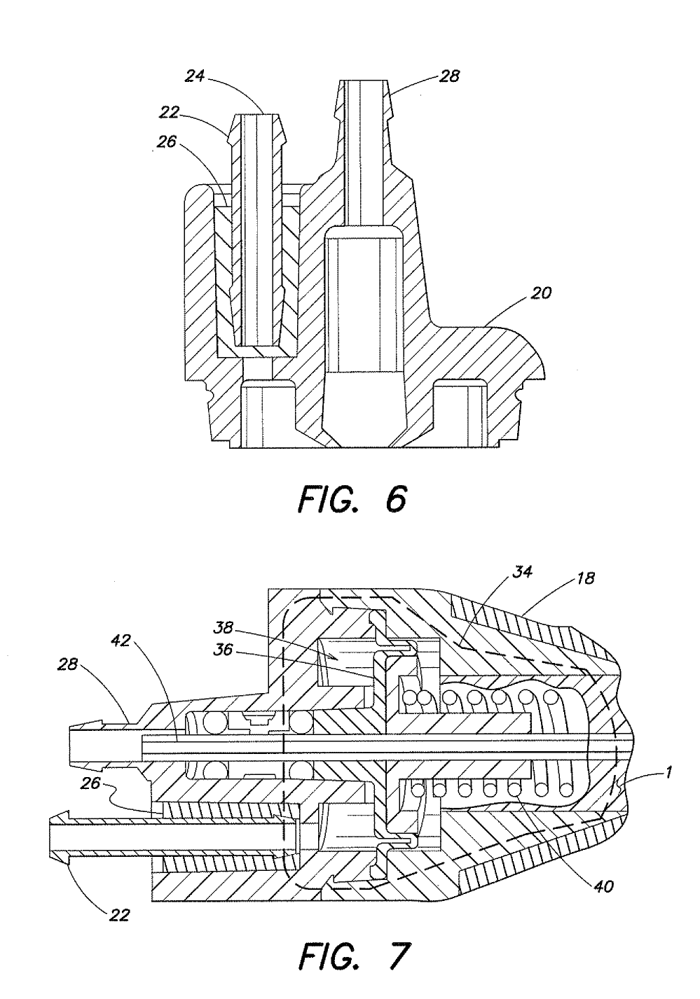

FIG. 6 is a cut-away view of FIG. 3 along line 6-6, and shows the relationship between pneumatic power port 20, which forms a part of housing 12, tubing connector 22, and length 26. By isolating rigid tubing connector 22 from the relatively stiff port 20 and housing 12 with the relatively pliable material length 26, any pneumatic power pulse vibrations transmitted will be dampened by connector 22 and length 26 before being transmitted via port 20 and housing 12 to a surgeon's fingers. In this way, the surgeon will have a more pleasant, stable, and less fatiguing experience, than known in the prior art.

FIG. 7 is a cut-away view of a portion of FIG. 2 along line 7-7, and shows an example of a pneumatic driver shown generally by dashed line 34. Pneumatic driver 34 is a known configuration for a reciprocating vit cutter, and includes a diaphragm 36, a cavity 38 for receiving pneumatic power pulses, and a return spring assembly 40. After a pneumatic power pulse causes diaphragm 36 to expand and move interior tubular cutter 42 across opening 30 (not shown in FIG. 7) to sever any tissue aspirated into exterior tubular cutter 15 (not shown in FIG. 7), return spring assembly 40 causes diaphragm 36 to contract and interior tubular cutter 42 to return to a starting position. Thus, it can be seen that one example of tissue manipulating structure 14 is a vit cutter tube set, including an interior tubular cutter 42 and an exterior tubular cutter 15, such that the interior and exterior cutters 42 and 15 cooperate to cut vitreous and other tissue when driven by the pneumatic driver 34. Diaphragm 36 of pneumatic driver 34 expands and contracts in response to pneumatic power pulses for activating the tissue manipulation structure 14. Those skilled in the art will appreciate that other drivers can be implemented for causing rotational or oscillating motion of cutters 42 and 15 or a completely different tissue manipulation structure could be used, such as a scissors or a morcellator.

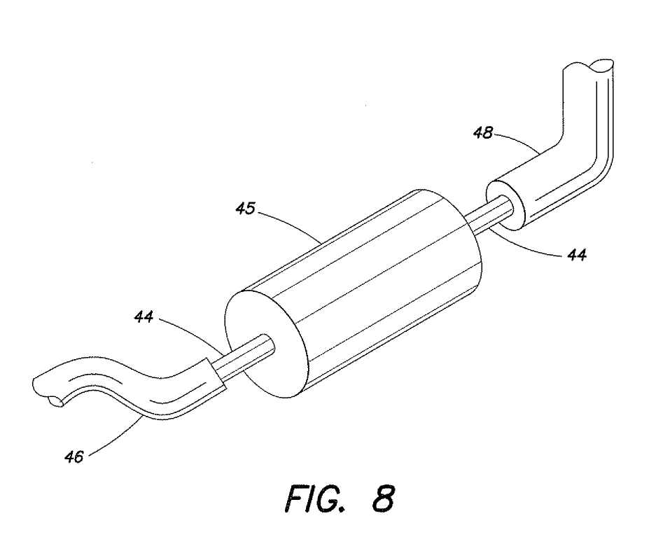

FIG. 8 shows an alternate embodiment where vibrations from pneumatic power pulses are dampened before the pulses reach a surgical instrument. A tubular length 45 of vibration dampening material surrounds one or more rigid tube connectors 44. Length 45 and connectors 44 may be formed of the same materials as length 26 and connector 22. In the example of FIG. 8, one connector 44 is attached to tubing 46 that supplies pneumatic power pulses, and another connector 44 is connected to tubing 48 that connects to a surgical instrument, not shown. Therefore, together length 45 and connectors 44 act similarly as described above with respect to length 26 and connector 22 to dampen vibrations caused by pneumatic power pulses.

Thus, there has been shown an invention for dampening vibrations caused by today's high speed pneumatic surgical instruments. The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

I claim:

1. An ophthalmic pneumatic surgical instrument comprising:
a housing;
a tissue manipulating structure extending from the housing;
a pneumatic driver contained within the housing for driving the tissue manipulating structure;
a pneumatic power port formed in the housing and for attachment to tubing that will supply pneumatic power pulses to the pneumatic driver; and
wherein the pneumatic power port includes a tubing connector with a lumen spanning a length of the tubing connector and a length of vibration dampening material surrounding at least a portion of the tubing connector for reducing vibrations created by the pneumatic power pulses transmitted through the pneumatic power port.

2. The instrument of claim 1 further including a layer of vibration dampening material surrounding at least a portion of the housing for reducing vibrations transmitted to a surgeon's fingers.

3. The instrument of claim 1, wherein the tissue manipulating structure is a vitreous cutter tube set including an interior tubular cutter and an exterior tubular cutter such that the interior and exterior cutters cooperate to cut vitreous and other tissue when driven by the pneumatic driver.

4. The instrument of claim 1, wherein the pneumatic driver includes a diaphragm that expands and contracts in response to the pneumatic power pulses for activating the tissue manipulation structure.

5. The instrument of claim 1, wherein the pneumatic power port tubing connector is formed of a material more rigid than a material forming the housing for reducing the amplitude of vibration caused by the pneumatic power pulses.

6. The instrument of claim 5, wherein the tubing connector is formed of a metal.

7. The instrument of claim 1, wherein the vibration dampening material is formed of rubber or synthetic rubber.

8. The instrument of claim 1, further including an extension handle connected to the housing for increasing the mass of the instrument to further dampen vibration from the pneumatic power pulses.

9. An ophthalmic vitreous cutter comprising:
a housing;
an interior tubular cutter and an exterior tubular cutter extending from the housing, such that the interior and exterior cutters cooperate to cut vitreous and other tissue during surgery;
a pneumatic driver contained within the housing for moving at least one of the inner and outer cutters to cut vitreous and other tissue;
an aspiration port formed in the housing;
a pneumatic power port formed in the housing and for attachment to tubing that will supply pneumatic power pulses to the pneumatic driver; and
wherein the pneumatic power port includes a tubing connector with a lumen spanning a length of the tubing connector and a length of vibration dampening material surrounding at least a portion of the tubing connector for reducing vibrations created by the pneumatic power pulses transmitted through the pneumatic power port.

10. The vitreous cutter of claim 9, further including a layer of dampening material surrounding at least a portion of the housing for reducing vibration energy transmitted to a surgeon's fingers.

11. The instrument of claim 9, wherein the pneumatic driver includes a diaphragm that expands and contracts in response to the pneumatic power pulses for activating one of the inner tubular cutter and the outer tubular cutter.

12. The instrument of claim 9, wherein the pneumatic power port tubing connector is formed of a material more rigid than a material forming the housing for reducing the amplitude of vibration caused by the pneumatic power pulses.

13. The instrument of claim 12, wherein the tubing connector is formed of a metal.

14. The instrument of claim 9, wherein the vibration dampening material is formed of rubber or synthetic rubber.

15. The instrument of claim 9, further including an extension handle connected to the housing for increasing the mass of the instrument to further dampen vibration from the pneumatic power pulses.

16. A pneumatic vibration dampening device for use with an ophthalmic surgical instrument comprising:
a body formed of vibration dampening material having a lumen spanning a length of the body; and
at least one rigid tubing connector attached to the body within a portion of the lumen.

17. The dampening device of claim 16, wherein the body is formed of rubber or synthetic rubber.

18. The dampening device of claim 16, wherein the tubing connectors are formed of a metal.
